(19) United States
(12) Patent Application Publication
(10) Pub. No.: US 2008/0044440 A1
(43) Pub. Date: Feb. 21, 2008

(54) Vaccine Formulated For Administration To Mucosa Of The Lungs

(76) Inventor: Margaret Dunkley, Elermore Vale (AU)

Correspondence Address: PEARNE & GORDON LLP, 1801 EAST 9TH STREET, SUITE 1200, CLEVELAND, OH, 44114-3108, US

(21) Appl. No.: 10/589,710

(22) PCT Filed: Feb. 18, 2005
(86) PCT No.: PCT/AU05/00214

§ 371 Date: Apr. 19, 2007

(30) Foreign Application Priority Data
Feb. 18, 2004 (AU) 2004900826

(51) Int. Cl. A61K 39/116; A61K 39/02; A61P 37/02; A61K 39/108

(52) U.S. Cl. 424/243.1; 424/234.1; 424/244.1; 424/256.1; 424/257.1; 424/260.1; 424/264.1

(57) ABSTRACT

There is disclosed vaccines formulated for administration to the mucosa of the lungs of a mammal, and the use thereof in methods for prophylaxis or treatment of an infection by at least one pathogenic microorganism. The vaccines comprise a cellular fraction of the microorganism that is essentially free of particulate matter and includes polyvalent soluble antigen from the microorganism.

### FIELD OF THE INVENTION

The present invention relates to a vaccine for inducing a protective immune response in the prophylaxis or therapy of acute or chronic infections.

### BACKGROUND OF THE INVENTION

Gram negative bacteria cause a variety of respiratory illnesses. For example, Non-typeable Haemophilus influenzae (NTHi) has been implicated in a range of infectious conditions including otitis media (Murphy, T. K., 1997) and infectious exacerbations in chronic bronchitis (Murphy, T. F., 2000). Streptococcus pneumoniae and Pseudomonas aeruginosa also cause infectious exacerbations in individuals with compromised lung function. P. aeruginosa is responsible for considerable morbidity and mortality in individuals with cystic fibrosis. Vaccines for Non-typeable H. influenzae or P. aeruginosa infection are not yet commercially available.

Antibiotics have been the treatment of choice for bacterial respiratory infections. However, bacterial pathogens can develop resistance to antibiotics. Antibiotics also have undesirable side-effects such as the elimination of “friendly” gut bacteria that are important for good health and the use of antibiotics in bronchitis patients has previously been reported to be of questionable benefit (Smucny J. S. et al., 2001).

Human anti-bacterial vaccines such as the conjugate vaccines against H. influenzae type B are predominantly injectable vaccines which induce systemic immune responses that provide some protection against respiratory infection. A S. pneumoniae vaccine is also available as an injectable formulation. Injection is by far the most common route of administration for human vaccines. Veterinary vaccines have also been delivered largely by injection, but nasal and aerosol routes of delivery have been used for the administration of some live viral veterinary vaccines (Deuter, A. et al., 1991).

A measles vaccine has been administered by an aerosol method in clinical trials (Fernandez-de Castro J. et al., 1997) and an oral vaccine is currently used for the prevention of polio. However, typically, particulate antigens at relatively high doses are used for oral immunisation (Cripps. A. W. et al., 1994). An oral vaccine utilising killed NTHi is known.

For effective vaccination, it is necessary to induce a protective immune response while avoiding the induction of immunological tolerance where the vaccinated host enters a state of being unable to respond to a pathogen with a protective response. The oral route of vaccination is particularly susceptible to tolerance induction (so-called oral tolerance). Indeed, suppression of both intestinal (Sugita-Konishi. Y. et al., 1992) and systemic immune responses (Melamed. D. and Friedman. A., 1993) have been demonstrated after antigen-feeding.

Routes of administration involving mucosal immunisation have been considered such as intra-nasal drops or aerosol application to the lung. These have been shown to be effective for protection against certain bacterial and viral infections when either inactivated or attenuated whole bacteria or viruses are utilised. However, in the same way that oral tolerance is a problem with oral immunisation, vaccination via the lung is also prone to the induction of tolerance (Sedgwick J. D. and Holt P. G., 1985). Lung vaccination may also induce allergic responses which can for instance complicate asthma or other such lung conditions, and possibly damage the lung which is a more serious issue in humans than farm animals due to the longer life span of humans. Accordingly, focus has generally been directed away from providing vaccines for delivery to such tissues.

### SUMMARY OF THE INVENTION

The present invention relates to the unexpected finding that protective immunity against at least some pathogenic microorganisms may be induced utilising relatively low amounts of soluble antigen from the microorganism(s) delivered to mucosa of the lung. This finding is surprising as it was expected that administration of soluble antigen provided by a cellular fraction of at least one pathogenic microorganism to mucosa of the lung would have induced immunological tolerance.

Accordingly, in one aspect of the present invention there is provided a method for prophylaxis or treatment of an infection in a mammal by at least one pathogenic microorganism, the method comprising administering an effective amount of a cellular fraction of the microorganism to mucosa of the lungs of the mammal for generating an immune response against the microorganism, wherein the cellular fraction is essentially free of particulate matter and includes polyvalent soluble antigen from the microorganism.

Infection by the microorganism may exacerbate a disease or condition in the mammal and the soluble antigen may therefore be administered to the mammal for prophylaxis or treatment of such diseases or conditions.

Hence, in another aspect of the present invention there is provided a method for prophylaxis or treatment of a disease or condition in a mammal associated with, or exacerbated by, infection by at least one pathogenic microorganism, the method comprising administering an effective amount of a cellular fraction of the microorganism to mucosa of the lungs of the mammal for generating an immune response against the microorganism, wherein the cellular fraction is essentially free of particulate matter and includes polyvalent soluble antigen from the microorganism.

In still another one aspect of the present invention there is provided a vaccine for inducing an immune response in a mammal against at least one pathogenic microorganism, wherein the vaccine is formulated for administration to mucosa of the lungs of the mammal and comprises a cellular fraction of the microorganism that is essentially free of particulate matter and includes polyvalent soluble antigen from the microorganism, together with a pharmaceutically acceptable carrier.

Typically, the cellular fraction(s) will be prepared from the whole microorganism(s) and will comprise cellular matter in addition to the soluble antigen. Preferably, the cellular fraction will comprise a non-particulate fraction of a sonicate of the, or each, microorganism respectively. Without being limited by theory, it is believed that the cellular matter comprises natural adjuvant from the microorganism(s) which promotes the immune response against the soluble antigen in the fraction.

Generally, the microorganism will be other than a virus and usually comprise a fungal, yeast or bacterial pathogen. Typically, the pathogen will be one that colonises the respiratory tract such as the lungs. However, it has been found that administration of the soluble antigen can result in the production of circulating antibody such that protection against infection of sites other than the respiratory tract may also be afforded by immunisation of the mucosa of the lung with the soluble antigen.

The soluble fraction may also be delivered to a site remote from the mucosa of the lung for priming the immune system and generation of an immune response against the microorganism(s) with subsequent administration of booster antigen to the mammal.

Accordingly, in a related embodiment there is provided a method for prophylaxis or treatment of an infection in a mammal by at least one pathogenic microorganism, the method comprising:
administering an effective amount of a cellular fraction of the microorganism to the mammal remotely from the mucosa of the lung to prime the immune system of the mammal for generation of an immune response against the microorganism, and
subsequently administering an effective amount of booster antigen to generate the immune response against the microorganism;
wherein the cellular fraction is essentially free of particulate matter and includes polyvalent soluble antigen from the microorganism.

Preferably, the microorganism will be a pathogenic bacteria. The bacteria will typically be selected from Non-typeable H. influenzae (NTHi), Streptococcus pneumoniae and Pseudomonas aeruginosa.

Preferably, the cellular fraction will induce a Th1 T-cell response in the mammal when administered to the mucosa of the lung. A Th1 response will typically be characterised by expression or up-regulation of a cytokine profile characteristic of a Th1 response. Up-regulation of the cytokine profile may be reflected by increased expression or activity of one or more cytokines characteristic of a Th1 T-cell response and/or suppressed expression or activity of one or more cytokines characteristic of a Th2 T-cell response.

A vaccine of the invention may be formulated with, or without, one or more added adjuvants. Preferably, the vaccine will not include any added adjuvant(s). However, when included in the vaccine, the added adjuvant(s) will desirably be selected to induce or promote a Th1 T-cell response and/or to suppress a Th2 T-cell response. However, up-regulation of a Th2 response is not excluded and indeed, adjuvant(s) may be selected to induce or enhance a Th2 response when deemed appropriate.

The phrase “mucosa of the lungs” wherever used herein is to be taken to encompass the mucosa of the trachea through to the mucosa of the pulmonary alveoli and to include the mucosa of the bronchi and bronchioles.

The mammal may be any mammal treatable with a method of the invention. For instance, the mammal may be a primate, a member of the rodent family such as a rat or mouse, or a member of the bovine, porcine, ovine or equine families. Typically, the mammal will be a human being.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of this application.

The features and advantages of the present invention will become further apparent from the following description of preferred embodiments.

### BRIEF DESCRIPTION OF THE FIGURES

FIG. 1 is a graph showing clearance of Haemophilus influenzae type b (Hib) infection following immunization with soluble Hib protein;

FIG. 2 is a graph showing clearance of S. aureus infection following immunization with soluble S. aureus protein; and

FIG. 3 is a graph showing clearance of P. aeruginosa infection following immunization with soluble P. aeruginosa protein.

### DETAILED DESCRIPTION OF PREFERRED EMBODIMENTS OF THE INVENTION

Vaccines of the invention find particular application in the prophylaxis or treatment of lung and respiratory tract infections. However, the invention is not limited thereto and circulating antibody generated as part of the immune response to the soluble antigen may provide protection against infections at other sites of the body including for example oral, nasal, oropharyngeal, nasalpharyngeal, pharyngeal, digestive tract, vaginal, urinary tract, kidney, eye and skin infections, as well as against systemic infections. P. aeruginosa for instance can colonise not only the respiratory tract but also infect the eye and skin. Systemic P. aeruginosa infections are also known.

Bacterial pathogens besides Non-typeable H. influenzae (NTHi), Streptococcus pneumoniae and Pseudomonas aeruginosa from which a cellular fraction may be prepared for a vaccine embodied by the invention include the stomach bacterium Helicobacter pylori, Haemophilus influenzae type b (Hib), Staphylococcus aureus, Staphylococcus albus, Chlamydia pneumoniae, Chlamydia trachomatis, Moraxella catarrhalis, Streptococcus pyrogenes, Chlostridium diptheriae, E. Coli species, and Mycoplasma species such as M. tuberculosis and M. genitalium.

Fungal pathogens include the candidiasis causing agent Candida albicans. Yeast pathogens from which the cellular fraction may be prepared include for instance Saccharomyces species. Similarly, non-particulate fractions comprising soluble antigen from microorganisms which normally colonise the respiratory tract or other sites in the body without causing disease, but which become opportunistic disease causing infections when conditions allow may also be utilised in vaccines and methods of the invention

Typically, the prepared cellular fraction will be administered to mucosa of the lung. However, administration to a site remote from the respiratory tract (eg. subcutaneously by injection) may also prime the immune system for generation of an immune response against the corresponding pathogenic bacteria with subsequent administration of booster antigen, such as whole killed bacteria of the same strain from which the soluble antigen was prepared, to for instance, mucosa of the respiratory tract or to mucosa of the lungs in particular. Rather than whole killed bacteria, the cellular fraction or a vaccine of the invention may provide the booster antigen for generating the immune response following the initial priming of the immune system against the microorganism.

The cellular fraction can be prepared by disrupting killed or viable microorganism(s), and filtering the resulting product to remove particulate matter. Any suitable method which achieves an appropriate level of cellular disruption may be employed including dissolution of cells utilising appropriate surfactants and agitation. Preferably, the microorganism(s) will be subjected to sonication, and the supernatant obtained following centrifugation of the sonicated preparation collected. The resulting cellular fraction comprises a polyvalent preparation of soluble antigens and other cellular debris. Again, without being limited by theory, it is believed the cellular debris includes natural adjuvant(s) produced by the sonication of the microorganism(s) which helps stimulate the protective immune response. The natural adjuvant(s) may for example comprise lipopolysaccaride and CpG oligodeoxynucleotides.

The sonication step may be repeated a number of times in order to obtain the desired degree of disruption of the microorganism and the release or generation of appropriate sized soluble antigen. The number of cycles and length of each may be determined by repeating the process a number of times employing a different number of cycles each time. Alternatively, or as well, the length of time the bacteria is sonicated may be varied. The filtrate is then collected and can tested for ability to produce a protective immune response utilising protocol as described below.

Generally, the soluble antigen and cellular debris from the microorganism(s) will be filterable through appropriate filters with a pore size of less than 0.60 μm and usually, through a pore size of less than 0.50 μm and more preferably, a pore size of about 30 μm or even about 0.20 μm or less. Accordingly, the resulting preparation will be substantially free of particulate matter from the microorganism(s).

While the cellular fraction will typically be utilised in the absence of added adjuvant, any appropriate adjuvant known in the art may be utilised in a vaccine of the invention to enhance the protective immune response generated. Suitable adjuvants include whole live, attenuated or killed yeast or bacteria other than that from which the soluble antigen is derived. Sonicates, antigens including individual antigens such as proteins or peptides, and/or homogenates of such other microorganisms may also be utilised. The microorganism used as the adjuvant or from which the adjuvant is derived will typically be one not normally associated with colonisation of the respiratory tract or lungs and preferably, will be a bacteria such as a lactic acid bacteria, Mycobacterium species or Bifidobacterium species. Lactobacillus species are particularly preferred such as L. acidophilus, L. fermentum, L. casei, L. plantarum and L. rhamnosus. Mycobacterium and Bifidobacterium species which may be used for adjuvanting purposes include M. vaccae and B. breve. Rather than being administered to the mucosa of the lung or respiratory tract, such adjuvant may be administered orally for generating the adjuvanting effect intestinally.

Examples of further adjuvants which may find use in vaccines and/or methods of the invention include for instance, the cross-linked form of polyidi(carboxylatophenoxy)lphosphazene (Adjumer)™, aluminium phosphate, gamma inulin/alum composite adjuvant, 1α,25-dihydroxycholecalciferol (calcitriol), calcium phosphate, cholera toxin B subunit, cholera toxin A1-subunit ProteinA D-fragment fusion protein (Lycke N., 1997), block copolymers such as CRL 1006 also known as block copolymer P1205, deoxycholic acid/alum complex, gerbu adjuvant, N-acetylglucosaminyl-(β14)-N-acetylmuramyl-L-alanyl-D-isoglutamine (GMDP) (Cas #70280-03-4), ISCOM(s) and E. coli labile enterotoxin protoxin (LT-OA). Adjuvants which stimulate T₀ type receptors of immune system cells are also expressly included.

The cellular fraction utilised for generation of the protective immune response and/or the vaccine itself may be freeze dried or lyophilized for later reconstitution utilising a physiologically acceptable buffer or fluid. A vaccine formulation of the invention may be in a powder or liquid form and may contain one or more anti-caking agents, isotonic agents, preservatives such as thimersal, stabilisers such as amino acids and sugar moieties, sweetening agents such as sucrose, lactose or saccharin, pH modifiers such as sodium hydroxide, hydrochloric acid, monosodium phosphate and/or disodium phosphate, a pharmaceutically acceptable carrier such as physiological saline, suitable buffers, solvents, dispersion media and isotonic preparations. Use of such ingredients and media for pharmaceutically active substances is well known. Except insofar as any conventional media or agent is incompatible with the soluble antigen and/or adjuvant utilised, their use in vaccines of the invention is specifically encompassed. Supplementary active agents such as one or more cytokines (eg. IL-2, IL-4, IL-12, IL-18, γ-IFN, and GM-CSF) for boosting the immune response or shifting the immune response towards a Th1 or Th2-like response can also be incorporated in the vaccine if desired.

Suitable adjuvants and pharmaceutically acceptable carriers useful in vaccine compositions of the present invention may for instance be found in handbooks and texts well known to the skilled addressee such as “Remington: The Science and Practice of Pharmacy (Mack Publishing Co., 1995)”, the contents of which is incorporated herein in its entirety by reference.

Effector T lympocytes are responsible for the cell-mediated immune responses of adaptive immunity and may be broadly categorised into three groups namely, cytotoxic T cells, Th1 cells and Th2 T-cells. Th1 cells stimulate antibacterial mechanisms of phagocytic cells such as neutrophils and macrophages, and release cytokines that attract such phagocytic cells to the site of infection. Th2 cells have a role in activating B-cells for generating antibodies against bacterial and other antigens. Cytokines typically secreted by Th1 cells include γ-interferon (γ-IFN), IL-12 and TNF-β. γ-IFN is the main phagocytic cell activating cytokine. TNF-β is directly cytotoxic for some cells. In contrast, Th2 cells secrete IL-4, IL-5, IL-10, IL-13, TGF-β and other cytokines. While both Th1 and Th2 cells both secrete IL-3, GM-CSF and for instance TNF-α, the overall cytokine profiles of each type of cell are different. Accordingly, a Th1 response can be detected by upregulated secretion of a cytokine characteristic of a Th1 immune response such as γ-IFN or IL-12. Similarly, a Th2 immune response may be characterised by upregulated expression of a cytokine characteristic of a Th2 response such as IL-4 or IL-10. The cellular fraction utilised in a vaccine of the invention will typically generate a Th1 immune response. The added adjuvant(s) of a vaccine formulation of the invention will also typically be selected for generating a Th1 immune response although adjuvants for generating a Th2 immune response may be utilised. In a particularly preferred embodiment, the cellular fraction and/or the added adjuvant will promote a Th1 immune response and/or suppress a Th2 immune response in the mammal.

A vaccine embodied by the invention will typically comprise the non-particulate matter from the microorganism(s) in an amount of between about 5% to about 80% w/w of the vaccine composition. As will be appreciated, the amount of soluble antigen in the vaccine will be such that an effective dosage will be delivered to the mammal for the generation of a protective immune response taking into account the proposed mode of delivery and added adjuvant(s) used in the composition. The dosage of the total non-particulate matter from the, or each, microorganism administered will typically be in a range of from about 10 μg/kg to about 70 μg/kg body weight of the recipient mammal, respectively. More preferably, the dosage will be in a range of from about 20 μg/kg to about 55 μg/kg, respectively. The optimum dosage of protein can be determined by administering different dosages of protein from each microorganism prepared as described herein to different groups of test animals, prior to subsequently infecting the animals in each group with the corresponding live microorganism, and determining the dosage levels required to achieve satisfactory clearance of the pathogen.

Non-typeable H. influenzae for instance, has been implicated in a range of infectious conditions including otitis media and in the exacerbation of pneumonia and chronic bronchitis as discussed above. Accordingly, a vaccine containing soluble antigen from this bacteria may be administered in accordance with the invention for the prophylaxis or treatment of those conditions. Vaccine comprising soluble antigen from H. influenzae may also be utilised in the prophylaxis or treatment of a lung diseases other than bronchitis such as cystic fibrosis, and as a treatment for preventing or ameliorating H. influenzae superinfection following infection by influenza virus or other virus.

Similarly, vaccines comprising S. pneumoniae and/or P. aeruginosa soluble antigen may also be utilised in the prophylaxis or treatment of respiratory infection in patients with compromised lung function, chronic bronchitis, pneumonia, cystic fibrosis and other lung diseases including asthma. Likewise, S. pneumoniae and/or P. aeruginosa vaccines can be used for the generation of a protective immune response to ameliorate or prevent S. pneumoniae and/or P. aeruginosa superinfection following influenza and other viral infections, particularly in the elderly.

A vaccine of the invention may be administered as a dry powder or liquid. Delivery may for example be achieved by aerosol inhalation, intranasal drops, intratracheal instillation, or as a spray. Devices for facilitating delivery of the vaccine are well known in the art and include metered dose inhalers (MDIs), dry powder inhalers (DPIs), and nebulisers including those which use ultrasonic energy or compressed air or other propellant to achieve atomisation. Propellants which may be used in MDIs include for instance chlorofluorocarbons (CFCs) such as trichlorofluorocarbon (CFC-11) and dichlorodifluorocarbon (CFC-12) and hydrofluoroalkanes.

In order that the nature of the present invention may be more clearly understood, preferred forms thereof will now be described with reference to the following non-limiting examples.

### EXAMPLE 1

### 1.1 Preparation of Soluble Antigen

Bacterial soluble antigen preparations were prepared by sonication of live bacteria followed by centrifugation to remove whole cells, and filtration to remove any remaining whole bacteria and particulate matter. The resulting preparation was bacteriologically sterile. More particularly, the sonication step comprised placing 10 ml of bacterial suspension in an 12 ml test tube and sonicating for 5, 10 or 20 cycles of 30 sec. on and 60 sec. off using a Soniprep 150 sonicator (MSE, United Kingdom) with a 5μ probe. The sonicated preparations were centrifuged at 10,000 g for 10 min and filtered through a 0.2 μm or 0.45 μm filter. Protein content was estimated utilising a Pierce BCA Protein Assay Kit (Pierce, Rockford, Ill., USA) following the manufacturer's instructions.

Male Dark Agouti rats were used in all studies utilising the soluble antigen preparations.

### EXAMPLE 2

2.1 Streptococcus pneumoniae Soluble Antigen Provides Protection. Against S. pneumoniae Infection

Rats (5 per group) were immunised by intra-tracheal instillation of phosphate buffered saline pH 7.2 (PBS) only (group A) or PBS containing 12.5 μg of S. pneumoniae soluble antigen preparation (group B) on days 0 and 14. On day 21, the rats were infected by intra-tracheal instillation of 4.5×10⁷ live S. pneumoniae in 50 μl of PBS. Rats were killed 4 hours later for sampling of broncho-alveolar lavage (BAL) and lung tissue. The total number of live bacteria in the airways and in the lung tissue was determined by serial dilution of BAL and lung homagenate (LH) samples. The mean bacteria number for each group was calculated. Effectiveness of immunisation with the soluble antigen preparation was determined by comparing the mean number of live bacteria in the antigen-immunised group with the mean number of live bacteria in the control (PBS immunised) group. The results are shown in Table 1.

TABLE 1
Live S. pneumoniae recovered from the lung

| | | | | % Clearance of |
|---|---|---|---|---|
| Rat | BAL CFU | | | bacteria* |
| group | (10⁶) | LH CFU (10⁶) | Total CFU (10⁶) | (Total CFU) |
| A (5) | 20.5 ± 8.2 | 7.8 ± 1.4 | 27.7 ± 8.6 | |
| B (5) | 5.0 ± 0.9 | 4.8 ± 1.8 | 9.8 ± 2.5 | 65 |

*Compared to control group A

The results show that protection against respiratory infection with S. pneumoniae was provided by immunisation with S. pneumoniae soluble antigen preparation delivered to the lung. The S. pneumoniae strain from which the soluble antigen was prepared and which was administered live to the rats has previously been described (Dunkley M. L. and Clancy R. L., 2001).

### EXAMPLE 3

### 3.1 Immunisation with Non-typeable Haemophilus influenzae (NTHi) Soluble Antigen Preparation: Effect on Subsequent Acute NTHi Respiratory Infection in Rats

Rats (5 per group) were immunised by intra-tracheal instillation of PBS (group A) or 25 μg NTHi soluble antigen preparation (group B) on days 0 and 14. On day 21 rats were infected by intra-tracheal instillation of 5×10⁸ live NTHi and killed 4 hours later for sampling. The number of live bacteria in the airways was determined by broncho-alveolar (BAL) wash, and in the lung tissue by analysis of lung homogenate. The results are shown in Table 2.

TABLE 2
Live NTHi recovered from the lung

| | | | | % Clearance of |
|---|---|---|---|---|
| Rat | BAL CFU | LH CFU | | bacteria* |
| Group | (10⁶) | (10⁶) | Total CFU (10⁶) | (Total CFU) |
| A | 4.6 ± 1.5 | 27 ± 4 | 31.6 ± 5.3 | |
| B | 0.49 ± 0.10 | 2.3 ± 0.4 | 2.8 ± 0.4 | 91 |
| | P = 0.028* | P = 0.0003* | | |

P values refer to comparison between groups A and B.
*Compared to control group A

The results show that the NTHi soluble antigen preparation when delivered as a 25 μg dose by the intra-tracheal route provides protection against subsequent NTHi respiratory infection. The H. influenzae strain from which the soluble antigen was prepared and which was administered live to the rats is a non-serotypeable, biotype 1 strain which has the gene for serotype b but does not express the b capsule. The strain (Hi289) has previously been described (Dunkley M. L. and Clancy R. L., 2001; Kyd. J., Dunkley M. and Cripps, A. W., 1995) where it was referred to as strain HI-CD. The strain was used for preparation of soluble antigen and challenge post immunisation with the soluble antigen in all following studies involving H. influenzae.

### 3.2 Optimal Dose Size of NTHi Soluble Antigen Preparation: Effect on Subsequent Acute NTHi Respiratory Infection in Rats

To determine the optimal dose of NTHi soluble antigen preparation delivered intra-tracheally for efficacy against a subsequent acute NTHi respiratory infection, rats (3 per group) were immunised on days 0 and 30 as shown in Table 3.

TABLE 3
Experimental protocol

| Rat group | IT day 0 | IT day 30 |
|---|---|---|
| A | PBS (diluent) | PBS |
| B | NTHi antigen prep. 1 μg | NTHi antigen prep. 1 μg |
| C | NTHi antigen prep. 5 μg | NTHi antigen prep. 5 μg |
| D | NTHi antigen prep. 25 μg | NTHi antigen prep. 25 μg |

The rats were infected by intra-tracheal instillation with live NTHi on day 37 and killed 4 hours later for sampling. The results are shown in Table 4.

TABLE 4
Live NTHi recovered from the lung

| | | | | % Clearance of |
|---|---|---|---|---|
| Rat | BAL CFU | | | bacteria* |
| group | (10⁶) | LH CFU (10⁶) | Total CFU (10⁶) | (Total CFU) |
| A | 1.8 ± 0.5 | 18.5 ± 4.2 | 20.4 ± 4.0 | |
| B | 1.2 ± 0.3 | 16.1 ± 5.4 | 17.3 ± 5.6 | 15 |
| C | 0.43 ± 0.11 | 6.6 ± 2.2 | 7.0 ± 2.3 | 66 |
| D | 0.28 ± 0.12 | 3.2 ± 0.9 | 3.5 ± 1.0 | 83 |

*Compared to control group A

The results show that NTHi soluble antigen preparation is effective in protecting against subsequent NTHi respiratory infection when delivered by the intra-tracheal route at concentrations as low as 5 μg although a 25 μg dose is more effective than a 5 μg dose. Only low clearance was observed for a dose of 1 μg when assessment is at 4 hours post-infection.

Significantly greater bacteria clearance may be observed utilising this dosage if rats were left longer after the induction of infection.

### 3.3 Comparison of Intra-nasal (i.n.) and Intra-tracheal (IT) Delivery of NTHi Soluble Antigen

Groups of rats were treated as set out in Table 5 to compare soluble NTHi antigen preparation delivered by the i.n. and IT routes.

TABLE 5
Experimental protocol

| Group | Treatment |
|---|---|
| A | PBS i.n. |
| B | NTHi antigen i.n. (5 μg antigen in 10 μL, 5 μL per nostril) |
| C | NTHi antigen IT (5 μg antigen in 50 μL) |

Rats were given a single dose of soluble antigen preparation or PBS on day 0 and challenged intra-tracheally with live NTHi on day 18. The results are shown in Table 6.

TABLE 6
Live NTHi recovered from lungs

| | | | | % Clearance of |
|---|---|---|---|---|
| | BAL CFU | LH CFU | Total CFU | bacteria* |
| Group | (10⁶) | (10⁶) | (10⁶) | (Total CFU) |
| A (PBS i.n.) | 7.7 ± 2.2 | 31.2 ± 7.5 | 38.9 ± 7.5 | |
| B (NTHi Ag i.n.) | 9.5 ± 1.9 | 19.4 ± 4.6 | 28.9 ± 6.0 | 26 |
| C (NTHi Ag IT) | 4.6 ± 0.95 | 18.3 ± 3.9 | 23.0 ± 4.4 | 41 |

*Compared to control group A.

The results show that intra-tracheal delivery of NTHi soluble antigen is more effective than intra-nasal delivery against subsequent NTHi challenge.

### 3.4 Subcutaneous (SC) Immunisation with NTHi Soluble Antigen Preparation.

To determine whether NTHi soluble antigen preparation delivered by the subcutaneous route and intra-tracheal boosting with whole killed NTHi can provide protection against subsequent NTHi respiratory infection, groups of rats were treated as set out in Table 7. The results are shown in Table 8.

TABLE 7
Experimental protocol

| Group (SC/IT boost) | Treatment |
|---|---|
| A | PBS SC, PBS IT |
| B | PBS SC, 4 × 10⁶ killed NTHi IT |
| C | NTHi antigen 5 μg SC, 4 × 10⁶ killed NTHi IT |
| D | NTHi antigen 25 μg SC, 4 × 10⁶ killed NTHi IT |

TABLE 8
Live NTHi recovered from lungs

| | | | | % Clearance of |
|---|---|---|---|---|
| Group | BAL CFU | LH CFU | Total CFU | bacteria* |
| (SC/IT boost) | (10⁶) | (10⁶) | (10⁶) | (Total CFU) |
| A PBS/PBS | 4.3 ± 0.9 | 7.3 ± 1.6 | 12.0 ± 1.9 | |
| B PBS/NTHi | 3.6 ± 1.3 | 5.7 ± 0.5 | 8.4 ± 1.5 | 30 |
| C 5 NTHi | 3.0 ± 1.1 | 4.7 ± 1.9 | 7.7 ± 2.7 | 36 |
| Ag/NTHi | | | | |
| D 25 NTHi | 2.2 ± 1.0 | 3.8 ± 1.2 | 6.6 ± 2.2 | 45 |
| Ag/NTHi | | | | |

*Compared to control group A.

The results show a degree of enhanced protection is afforded by subcutaneous delivery of NTHi soluble antigen when followed by IT administration of whole killed NTHi. The level of protection is less than that provided by intra-tracheal immunisation with soluble antigen (see for instance Example 3.2).

### 3.5 NTHi Soluble Antigen Delivered Subcutaneously or Intra-tracheally.

To determine whether NTHi soluble antigen preparation is protective when delivered only by the subcutaneous (SC) route without subsequent intra-tracheal (IT) boosting with whole killed NTHi, and the degree of protection compared to that provided by IT immunisation with soluble NTHi antigen, groups of 6 rats were subjected to subcutaneous or IT delivery of NTHi soluble antigen preparation. Both the protocol used and the results are shown in Table 9.

TABLE 9
Experimental protocol and results

| | | | | | % Clearance |
|---|---|---|---|---|---|
| | | BAL CFU | LH CFU | Total CFU | of bacteria* |
| Group | Treatment | (10⁶) | (10⁶) | (10⁶) | (Total CFU) |
| A | PBS IT | 8.1 ± 2.3 | 19.0 ± 3.3 | 27.1 ± 4.8 | |
| B | NTHi | 0.7 ± 0.2 | 4.1 ± 1.8 | 4.8 ± 2.0 | 82 |
| | antigen IT | | | | |
| C | PBS SC | 9.3 ± 3.6 | 20.2 ± 1.5 | 29.5 ± 4.4 | |
| D | NTHi | 13.9 ± 3.1 | 20.6 ± 2.7 | 34.5 ± 5.5 | 0 |
| | antigen | | | | |
| | SC | | | | |

*Compared to control group A or C.

The IT and SC doses were administered on days 0 and 14. The rats were subsequently infected intra-tracheally with live NTHi on day 21. As can be seen from Table 9, SC administration of NTHi antigen had no protective effect when administered in the absence of an intra-tracheal boost. In contrast, good protection was provided by IT administration of NTHi soluble antigen.

### 3.6 Intra-lumenal Dosing Prior to IT Immunisation with NTHi Soluble Antigen

To determine if protection is afforded by intestinal immunisation when NTHi soluble antigen preparation is injected directly into the intestinal lumen (as a correlate of an enteric-coated oral dose), and whether a combination of intestinal an IT immunisation gives better protection against NTHi respiratory infection, groups of 6 rats were treated as set out in Table 10.

TABLE 10
Experimental protocol

| | Intra-lumenal (IL) | Intra-tracheal (IT) |
|---|---|---|
| Group | immunisation | immunisation |
| A | PBS | PBS |
| B | PBS | NTHi antigen prep. (25 μg) |
| C | NTHi antigen (250 μg) | PBS |
| D | NTHi antigen (250 μg) | NTHi antigen prep. (25 μg) |

The IL immunisation was performed on day 0. The IT immunisation was performed on day 14. The rats were subsequently infected intra-tracheally with live NTHi and killed on day 21 and the results are shown in Table 11.

TABLE 11
Intestinal and intra-tracheal administration
of NTHi soluble antigen preparation.

| | | | | % Clearance |
|---|---|---|---|---|
| | BAL CFU | LH CFU | Total CFU | of bacteria* |
| Group (IL/IT) | (10⁶) | (10⁶) | (10⁶) | (Total CFU) |
| A (PBS/PBS) | 5.8 ± 1.8 | 74.9 ± 33.9 | 80.7 ± 35.7 | |
| B (PBS/NTHi) | 4.0 ± 1.0 | 19.3 ± 3.1 | 23.3 ± 3.4 | 71 |
| C (NTHi/PBS) | 32.1 ± 11.0 | 46.2 ± 13.9 | 78.3 ± 23.8 | 3 |
| D | 17.6 ± 4.7 | 58.3 ± 10.7 | 75.8 ± 13.0 | 6 |
| (NTHi/NTHi) | | | | |

*Compared to control group A.

Single dose IT administration of NTHi soluble antigen provided a 71% clearance of infection. In contrast, a single dose of NTHi IL did not provide any protection and may have induced some tolerance. A single dose of NTHi soluble antigen preparation IL followed by IT administration of NTHi soluble antigen preparation also did not provide any protection further indicating tolerance induction by the IL dose. A dose of 250 μg of the NTHi soluble antigen preparation was administered IL which is ten-fold higher than the IT dose utilised. This was to minimise the likelihood of the vaccine being unavailable due to being caught up in digested food in the gut.

### 3.7 IT Immunisation Followed By IL Immunisation with NTHi Soluble Antigen

To determine whether IL immunisation with NTHi soluble antigen preparation following IT immunisation with NTHi soluble antigen preparation gives better protection than IT immunisation alone, groups of 6 rats were treated as set out in Table 12.

TABLE 12
Experimental protocol

| Group | IT | IL |
|---|---|---|
| A | PBS | PBS |
| B | NTHi antigen 25 μg | NTHi antigen 250 μg |
| C | PBS | NTHi antigen 250 μg |
| D | NTHi antigen 25 μg | PBS |

IT immunisation with NTHi soluble antigen preparation was performed on day 0. IL immunisation was performed on day 14. Rats were subsequently infected intra-tracheally with live NTHi and killed on day 28 and the results are shown in Table 13.

TABLE 13
Intestinal administration of NTHi soluble antigen preparation followed
by intra-lumenal administration of the antigen.

| | | | | % Clearance |
|---|---|---|---|---|
| | BAL CFU | LH CFU | Total CFU | of bacteria* |
| Group (IT/IL) | (10⁶) | (10⁶) | (10⁶) | (Total CFU) |
| A (PBS/PBS) | 6.7 ± 3.1 | 11.0 ± 4.6 | 17.6 ± 6.7 | |
| B (NTHi/NTHi) | 1.3 ± 0.3 | 5.1 ± 1.3 | 6.4 ± 1.5 | 64 |
| C (PBS/NTHi) | 1.0 ± 0.4 | 7.6 ± 2.2 | 8.6 ± 2.1 | 51 |
| D (NTHi/PBS) | 1.3 ± 0.6 | 6.8 ± 1.9 | 8.2 ± 1.7 | 53 |

*Compared to control group A.

As in the above studies, the single IT dose provided protection against subsequent NTHi respiratory infection (53% clearance). Unlike the results in Example 3.7, the single IL dose also provided protection (51% clearance). The NTHi antigen preparation was not filtered prior to use in this instance, and had an opaque appearance suggesting the presence of particulate antigen. From the results, it is apparent that protection can be provided by intra-lumenal immunisation with the soluble antigen preparation if it is unfiltered and this is likely to be due to the presence of particulate material in the administered dose. The best protection was provided by the combined IT/IL dosing (64% clearance).

### 3.8 NTHi Soluble Antigen Delivered IT or to Gut Lumen

To provide a repeat determination of whether soluble NTHi antigen preparation delivered to gut lumen by intra-lumenal (IL) administration protects against subsequent respiratory infection by live NTHi, and to determine whether prior administration of antigen IL affects the protection obtained by NTHi soluble antigen preparation delivered IT, 6 rats per group were treated as set out in Table 14.

TABLE 14
Experimental protocol

| | | | | | % |
|---|---|---|---|---|---|
| | | | | | Clearance |
| | Treatment | BAL CFU | LH CFU | Total CFU | of bacteria* |
| Group | ID/IT | (10⁶) | (10⁶) | (10⁶) | (Total CFU) |
| A | PBS/PBS | 6.7 ± 2.1 | 14.7 ± 2.2 | 21.4 ± 3.4 | |
| B | NTHi Ag/ | 1.4 ± 0.4 | 4.9 ± 2.7 | 6.4 ± 3.1 | 70 |
| | NTHi Ag | | | | |
| C | NTHi Ag/ | 9.3 ± 1.8 | 19.9 ± 4.3 | 29.1 ± 4.8 | 0 |
| | PBS | | | | |
| D | PBS/NTHi | 1.3 ± 0.7 | 4.2 ± 1.5 | 5.5 ± 2.1 | 74 |
| | Ag | | | | |

*Compared to control group A.

Again, IL administration of NTHi soluble antigen was found not to provide protection against subsequent respiratory infection with NTHi soluble antigen. In contrast, IT administration of NTHi soluble antigen provided protection. Prior IL administration of antigen did not affect the level of protection provided by IT administered NTHi soluble antigen. (For total CFU data, values for B and D were significantly lower than A, with P=0.0148 and 0.0024 respectively. B and D were not significantly different from each other).

Overall, the preferred route of immunisation with NTHi soluble antigen preparation is administration to the lungs. This is a accomplished by intra-tracheal delivery in rats and may be accomplished in humans by, for example, aerosol delivery of the soluble antigen.

### EXAMPLE 4

### 4.1 Preparation of Soluble Antigen from P. aeruginosa Strain 385 (Pa385)

P. aeruginosa strain 385 was grown on nutrient agar, harvested and three preparations subjected to sonication treatment of 5, 10 or 20 cycles as described in Example 1.1. Protein estimations were performed and each preparation was adjusted to 5 μg protein/ml. The Pa385 strain used for preparation of the soluble antigen and challenge post immunisation with soluble antigen in all the following examples is a serotype 2 strain which has previously been described (Dunkley M. L., and Clancy R. L., 2001; Cripps A. W., Dunkley M. L., and Clancy R. L., 1994).

### 4.2 Intra-tracheal Immunisation with Pa385 Soluble Antigen

Groups of 6 rats were immunised IT with Pa385 soluble antigen preparation obtained from the 20 cycle sonication treatment or with vehicle (PBS) on days 0 and 14. Rats were subsequently infected by intra-tracheal instillation of Pa385 and killed on day 21. The results are shown in Table 15.

TABLE 15
Intra-tracheal immunisation with Pa385 soluble
antigen preparation (20 cycle)

| | | | % Clearance of |
|---|---|---|---|
| Group | Dosed IT with: | Total CFU (10⁶) | bacteria* (Total CFU) |
| A | PBS | 615 ± 124 | |
| B | 25 μg Pa antigen | 273 ± 59 | 56 |
| C | 5 μg Pa antigen | 284 ± 121 | 54 |
| D | 1 μg Pa antigen | 381 ± 102 | 38 |

*Compared to control group A

The results show that IT immunisation with Pa385 soluble antigen prepared by the 20 cycle sonication treatment provides reasonable protection against subsequent acute Pa385 respiratory infection.

### 4.2 Intra-tracheal Immunisation with Soluble Pa385 Antigen (10 Cycle)

Groups of rats were immunised IT on days 0 and 14 with Pa385 soluble antigen preparation obtained from a 10 cycle sonication treatment or with vehicle (PBS). Rats were infected by intra-tracheal instillation of live Pa385 and killed on day 21. The results are shown in Table 16.

TABLE 16
Intra-tracheal immunisation with Pa385 soluble
antigen preparation (10 cycle)

| | | | % Clearance of |
|---|---|---|---|
| Group | Dosed IT with | Total CFU (10⁶) | bacteria* (Total CFU) |
| A | PBS | 575 ± 126 | |
| B | 25 μg Pa antigen | 87 ± 20 | 85 |
| C | 5 μg Pa antigen | 126 ± 45 | 78 |
| D | 1 μg Pa antigen | 441 ± 121 | 23 |

*Compared to control group A

The results show that immunisation IT with Pa385 soluble antigen prepared by the 10 cycle sonication treatment provided better protection against subsequent acute Pa385 infection than immunisation with 20 cycle Pa385 antigen (see Example 4.2).

### 4.3 Intra-tracheal Immunisation with Pa385 Soluble Antigen (5 Cycle)

Groups of rats were immunised IT on days 0 and 14 with Pa385 soluble antigen preparation or vehicle (PBS). Rats were subsequently infected by intra-tracheal installation of live Pa385 and killed on day 21. The results are shown in Table 17.

TABLE 17
Intra-tracheal immunisation with Pa385 soluble
antigen preparation (5 cycle)

| | | | % Clearance of |
|---|---|---|---|
| Group | Dosed IT with | Total CFU (10⁶) | bacteria* (Total CFU) |
| A | PBS | 778 ± 106 | |
| B | 25 μg Pa antigen | 149 ± 39 (81% | 81 |
| | | cleared) | |
| C | 5 μg Pa antigen | 277 ± 73 (64% | 64 |
| | | cleared) | |
| D | 1 μg Pa antigen | 440 ± 94 (43% | 43 |
| | | cleared) | |

*Compared to control group A.

The results show that immunisation with Pa385 soluble antigen prepared by the 5 cycle sonication treatment provides less protection against acute respiratory Pa385 infection then immunisation with Pa385 soluble antigen prepared by the 10 cycle sonication treatment but more protection than immunisation with Pa385 soluble antigen preparation obtained from 20 cycles of the sonication treatment. Pa385 soluble antigen preparation obtained using the 10 cycle sonication treatment was used in all further experiments involving Pa385.

### 4.4 Subcutaneous (SC) Immunisation with Pa385 Soluble Antigen

Groups of rats were immunised SC on days 0 and 14 with Pa385 soluble antigen preparation or vehicle (PBS). Rats were subsequently infected by intra-tracheal installation with live Pa385 and killed on day 21. The results are shown in Table 18.

TABLE 18
Subcutaneous immunisation with soluble Pa385 antigen preparation

| | | | % Clearance of |
|---|---|---|---|
| Group | Dosed SC with | Total CFU (10⁶) | bacteria* (Total CFU) |
| A | PBS | 179 ± 36 | |
| B | 25 μg Pa antigen | 37 ± 9 | 79 |
| C | 5 μg Pa antigen | 108 ± 58 | 40 |
| D | 1 μg Pa antigen | 92 ± 34 | 49 |

*Compared to control group A.

The results show that SC immunisation with Pa385 soluble antigen preparation does not provide as much protection as IT immunisation with Pa385 soluble antigen preparation.

### 4.5 Immunisation with Pa385 Soluble Antigen Provides Protection against Pa Strains NCTC 11440 and NCTC 11446

Groups of rats were immunised IT on days 0 and 14 with 5 μg of Pa385 soluble antigen preparation or vehicle (PBS). Rats were subsequently infected by intra-tracheal instillation with live Pa385 strains available from the National Collection of Type Cultures (NCTC), PHLS Central Public Health Laboratory, London, United Kingdom under accession numbers NCTC 11440 and NCTC 11446. The strains are also available from the American Type Culture Collection (ATCC), Manassas, Va., United States, under accession numbers ATCC 33348 and ATCC 33354, respectively. The results are shown in Table 19.

TABLE 19
Intra-tracheal immunisation with soluble antigen preparation from
P. aeruginosa strains NCTC 11440 and NCTC 11446

| | | | | % Clearance of |
|---|---|---|---|---|
| | Immunised | | Total CFU | bacteria* |
| Group | with: | Challenged with: | (10⁶) | (Total CFU) |
| A | PBS | NCTC 11440 | 210 ± 58 | |
| B | Pa antigen | NCTC 11440 | 93 ± 33 | 56 |
| C | PBS | NCTC 11446 | 98 ± 20 | |
| D | Pa antigen | NCTC 11446 | 37 ± 6 | 62 |

*Compared to control group A or C.

The results show IT immunisation with Pa385 soluble antigen preparation provided protection against subsequent acute respiratory infection with Pa385 strains NCTC 11440 and NCTC 11446.

### 4.6 Intra-tracheal Immunisation with Pa385 Soluble Antigen against P. aeruginosa Strains NCTC 11450 and NCTC 11451

Groups of rats were immunised IT on days 0 and 14 with 5 μg of Pa385 soluble antigen preparation or with vehicle (PBS). Rats were subsequently infected by infra-tracheal installation with Pa385 strains and killed on day 21. THE Pa385 strains utilised are available from the NCTC under accession numbers NCTC 11450 and NCTC 11451. The strains are also available from the ATCC under accession numbers ATCC 33358 and ATCC 33359. The results are shown in Table 20.

TABLE 20
Live bacteria recovered from lungs

| | | | | % Clearance of |
|---|---|---|---|---|
| | Immunised | | Total CFU | bacteria* |
| Group | with: | Challenged with: | (10⁶) | (Total CFU) |
| A | PBS | NCTC 11450 | 27.8 ± 8.1 | |
| B | Pa antigen | NCTC 11450 | 40.8 ± 7.7 | 0 |
| C | PBS | NCTC 11451 | 75.6 ± 7.5 | |
| D | Pa antigen | NCTC 11451 | 35.7 ± 13.7 | 53 |

*Compared to control group A

The results show that IT immunisation with Pa385 soluble antigen preparation provided protection against subsequent acute respiratory infection with Pa strain NCTC 11451 but no protection was provided against strain NCTC 11450.

### 4.7 Oral Immunisation with Pa385 Soluble Antigen

Groups of 6 rats were immunised by gavage on days 0 and 14 with 10, 50 or 250 μg Pa385 soluble antigen preparation or vehicle (PBS). Rats were subsequently infected by intra-tracheal installation with live Pa385 and killed on day 21. The results are shown in Table 21.

TABLE 21
Oral immunisation results

| | | | % Clearance of |
|---|---|---|---|
| Group | Immunised with: | Total CFU (10⁶) | bacteria* (Total CFU) |
| A | PBS | 1104 ± 220 | |
| B | Pa antigen 10 μg | 1194 ± 196 | 0 |
| C | Pa antigen 50 μg | 864 ± 182 | 22 |
| D | Pa antigen 250 μg | 1090 ± 205 | 1 |

*Compared to control group A

The results show that no significant protection was provided by Pa385 soluble antigen preparation delivered orally. Event though a ten-fold higher dose than that which provides protection by the IT route was administered (allowing for 90% destruction of the antigen in the stomach) no protection was observed. This suggests either total destruction of antigen, failure of soluble antigen to immunise the intestinal lymphoid tissue or induction of oral tolerance.

### 4.8 Induction of Specific Antibodies by Intra-tracheal Immunisation with Pa385 Soluble Antigen

Groups of rats were immunised IT on days 0 and 14 with 1, 5 or 25 μg of Pa385 soluble antigen preparation or vehicle (PBS). Rats were subsequently infected by intra-tracheal installation with live Pa385 and killed on day 21. Antibody levels were assessed employing an ELISA assay using 96-well polysorp microlitre plates coated with soluble antigen prepared as described above. Dilutions of serum or BAL samples were added to the plates and bound antibody detected with anti-rat IgG, IgG1, IgG2, IgA or IgM antibody conjugated to horse-radish peroxidase (Nordic, The Netherlands). The bound conjugated antibody was detected by addition of tetramethyl benzidine (TMB), the reaction stopped with sulfuric acid and the optical density measured at 450 mm with a 690 nm background filter using a BioRad Model 450 Plate Reader (BioRad Australia). The results are shown in Table 22.

TABLE 22
Intra-tracheal installation results

| Group | BAL IgM | BAL IgA | BAL IgG | BAL IgG1 | BAL IgG2b |
|---|---|---|---|---|---|
| PBS | 0.09 ± 0.03 | 0.12 ± 0.09 | 1.4 ± 0.6 | 0.5 ± 0.2 | 2.1 ± 0.8 |
| 1 μg Pa Ag | 2.65 ± 0.72 | 2.31 ± 0.52 | 84.6 ± 20.7 | 23.2 ± 7.4 | 369.0 ± 112.5 |
| 5 μg Pa Ag | 2.12 ± 0.29 | 5.06 ± 0.85 | 409.4 ± 51.1 | 57.5 ± 16.2 | 395.9 ± 85.8 |
| 25 μg Pa Ag | 1.78 ± 0.28 | 4.49 ± 1.01 | 401.5 ± 80.2 | 186.2 ± 23.1 | 620.2 ± 83.8 |
| Group | Serum IgM | Serum IgA | Serum IgG | Serum IgG1 | Serum IgG2b |
| PBS | 6.3 ± 1.4 | 6.0 ± 1.9 | 115.4 ± 25.5 | 23.4 ± 8.0 | 188 ± 105 |
| 1 μg Pa Ag | 275.7 ± 62.3 | 43.3 ± 9.8 | 4758 ± 116 | 1187 ± 261 | 2741 ± 1043 |
| 5 μg Pa Ag | 194.1 ± 32.3 | 145.3 ± 44.0 | 8129 ± 1766 | 3283 ± 456 | 2973 ± 1632 |
| 25 μg Pa Ag | 272.6 ± 53.3 | 17.3 ± 5.5 | 52038 ± 3356 | 8634 ± 3360 | 8456 ± 450 |

The results show that IT immunisation with Pa385 soluble antigen preparation induced P. aeruginosa specific IgA and Ig antibody responses that are measurable in both the airways (BAL assay) and serum.

As for NTHi soluble antigen preparation, the preferred route of administration P. aeruginosa soluble antigen preparation is to the lungs. This was accomplished in laboratory rodents by intra-tracheal installation and may be accomplished in humans by inhalation of the soluble antigen preparation in an aerosol or in dry powder form.

### EXAMPLE 5

### 5.1 Protection Against Systemic Haemophilus influenzae Type b (Hib) Infection

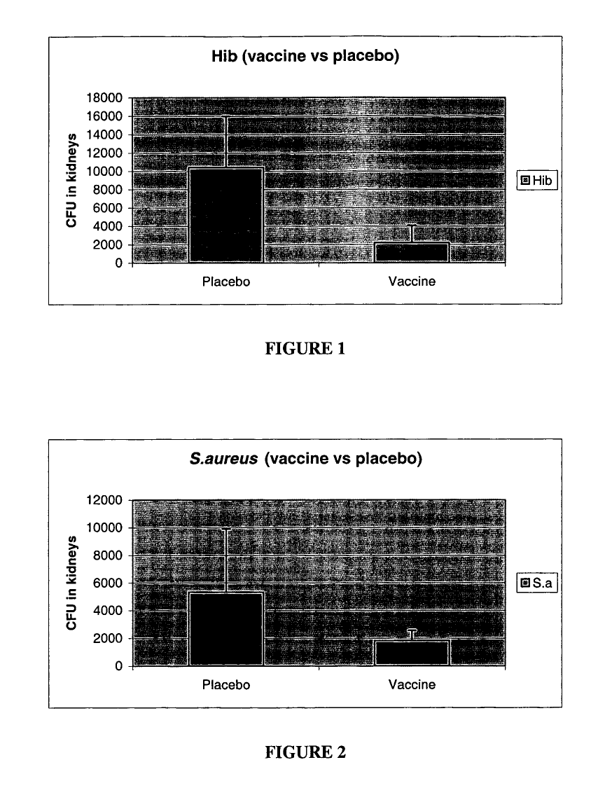

Rats were immunized by two intra-tracheal doses of placebo (PBS) or Hib vaccine preparation (25 micrograms soluble Hib protein in PBS) on days 0 and 14. On day 21 rats were infected by intra-venous instillation of live Hib (10⁶ CFU in 0.2 mL) and the infection level in the kidneys evaluated 4 h later. Briefly, the kidneys from the rats were homogenised in 10 ml PBS. Twenty microlitres of serial ten-fold dilutions of homogenate were plated out on chocolate agar plats and colonies counted after 24 hours incubation at 37° C. The total number of bacteria (colony forming units) in the kidneys was calculated. The protection provided by the Hib vaccine preparation was determined by calculation of the percentage of bacteria cleared in the vaccine group compared to the placebo group. The administration of Hib vaccine resulted in 79% clearance of the Hib infection compared to the placebo as shown in FIG. 1.

### 5.2 Protection against Systemic Infection with Staphylococcus aureus

Rats were immunized by two intra-tracheal doses of placebo (PBS) or S. aureus vaccine preparation (5 micrograms soluble S. aureus protein in PBS) on days 0 and 14. On day 21 rats were infected by intra-venous instillation of live S. aureus (10⁶ CFU in 0.2 mL) and the infection level in the kidneys evaluated 4 h later. Infection of the kidneys was assessed as described in Example 5.1 except that the ten-fold dilutions of homogenate were plated out on nutrient agar plates. The administration of the S. aureus vaccine resulted in 66% clearance of the S. aureus infection compared to the placebo as shown in FIG. 2.

### 5.3 Protection against Systemic Infection with Pseudomonas aeruginosa

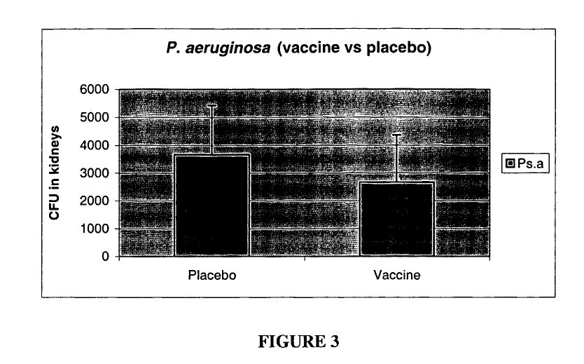

Rats were immunized by two intra-tracheal doses of placebo (PBS) or P. aeruginosa vaccine preparation (25 micrograms soluble P. aeruginosa protein) on days 0 and 14. On day 21 rats were infected by intra-venous instillation of live P. aeruginosa (10⁶ CFU in 0.2 mL) and the infection level in the kidneys evaluated 4 h later. The administration of the P. aeruginosa vaccine resulted in 27% clearance of the P. aeruginosa infection compared to the placebo as shown in FIG. 3.

Overall, the results of the present study show that soluble antigen preparations made by sonication and filtration to remove particulate matter can be used as vaccines for protection against respiratory bacterial infection by Heamophilus influenzae type b (Hib), Non-typeable Haemophilus influenzae (NTHi), S. pneumoniae and/or P. aeruginosa. The examples further show that administration of soluble antigen preparation to mucosa of the respiratory tract produces a protective immune response not immunological tolerance.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

1. Cripps, A. W., Dunkley, M. L., Clancy, R. L. (1994) Mucosal and systemic immunizations with killed Pseudomonas aeruginosa protect against respiratory infection in rats. Infect. Immun. 62, 1427-1436.
2. Deuter, A., Southee, D. J., Mockett, A. P. (1991) Fowlpox virus: pathogenicity and vaccination of day-old chickens via the aerosol route. Res. Vet. Sci. 50, 362-364.
3. Dunkley, M. L. and Clancy R. I., 2001. Elsevier International Congress Series 1219, pp. 581-585.
4. Fernandez-de Castro, J., Kumate-Rodriguez, J., Sepulveda, J., Ramirez-Isunza, J. M. and Valdespino-Gomez, J. L. (1997) Measles vaccination by the aerosol method in Mexico. Salud Publica de Mexico 39, 53-60.
5. Kyd, J., Dunkley M. L., and Cripps A. W. (1995) Infect and Immunity 63, pp. 2931-2940.
6. Melamed, D. and Friedman, A. (1993) Modification of the immune response by oral tolerance: Antigen requirements and interaction with immunogenic stimuli. Cellular Immunology 146, 412-420.
7. Murphy T F (2000) Haemophilus influenzae in chronic bronchitis. Sem. Resp. Infect. 15, pp. 41-51.
8. Murphy T K (1997). Mechanisms of recurrent otitis media: importance of the immune response to bacterial surface antigens. Ann. N. Y. Acad. Sci. 830, pp. 353-360.
9. Sedgwick, J. D. and Holt, P. G. (1985) Down-regulation of immune responses to inhaled antigen: studies on the mechanism of induced suppression. Immunology 56, 635-42.
10. Smucny J, Fahey T, Becker L, Glazier R. Antibiotics for acute bronchitis (Cochrane Review). In: The Cochrane Library, 3, 2001. Oxford: Update Software.
11. Sugita-Konishi, Y., Smart, C. J., Trejdosiewicz, L. K. (1992) Regulation of intestinal immunoglobulin production in response to dietary ovalbumin. Int. Arch. Allergy Immunol. 98, 64-69.

1. A vaccine for inducing an immune response in a mammal against at least one pathogenic microorganism, wherein the vaccine is formulated for administration to mucosa of the lungs of the mammal and comprises a cellular fraction of the microorganism that is essentially free of particulate matter and includes polyvalent soluble antigen from the microorganism, together with a pharmaceutically acceptable carrier.

2. A vaccine according to claim 1 wherein the cellular fraction comprises cellular matter and the polyvalent soluble antigen.

3. A vaccine according to claim 1 wherein the vaccine is formulated without any added adjuvants.

4. A vaccine according to claim 1 wherein the vaccine is formulated with one or more added adjuvants.

5. A vaccine according to claim 4 wherein the adjuvant is selected to promote a Th1 T-cell immune response and/or to suppress a Th2 T-cell immune response in the mammal.

6. A vaccine according to claim 1 wherein the cellular fraction is prepared from the whole said microorganism.

7. A vaccine according to claim 1 wherein the cellular fraction is a sonicate.

8. A vaccine according to claim 1 wherein the cellular fraction is filterable through a filter with a pore size of less than 0.60 μm.

9. A vaccine according to claim 8 wherein the pore size is less than 0.20 μm.

10. A vaccine according to claim 1 wherein the microorganism is a microorganism that colonises the lung or respiratory tract.

11. A vaccine according to claim 1 wherein the protective immune response is a systemic immune response.

12. A vaccine according to claim 1 for the prophylaxis or treatment of an infection by the microorganism selected from the group consisting of lung, oral, nasal, oropharyngeal, nasalpharyngeal, pharyngeal, respiratory tract, digestive tract, vaginal, urinary tract, kidney, eye and skin infections.

13. A vaccine according to claim 12 wherein the infection is a lung or respiratory tract infection.

14. A vaccine according to claim 1 wherein the microorganism is a bacterial, fungal or yeast pathogen.

15. A vaccine according to claim 14 wherein the microorganism is a bacterial pathogen selected from the group consisting of Non-typeable H. influenzae, Moraxella catarrhalis, S. pneumoniae, P. aeruginosa, H. influenzae type b, H. pylori, S. aureus, S. albus, C. pneumoniae, C. trachomatis, S. pyrogenes, E coli species and Mycoplasma species.

16. A vaccine according to claim 15 wherein the bacterial pathogen is selected from Non-typeable H. influenzae, S. pneumoniae and P. aeruginosa.

17. A method for prophylaxis or treatment of an infection in a mammal by at least one pathogenic microorganism, the method comprising administering an effective amount of a cellular fraction of the microorganism to mucosa of the lungs of the mammal for generating an immune response against the microorganism, wherein the cellular fraction is essentially free of particulate matter and includes polyvalent soluble antigen from the microorganism.

18. A method according to claim 17 wherein the cellular fraction comprises cellular matter and the polyvalent soluble antigen.

19. A method according to claim 17 wherein the vaccine is formulated without any added adjuvants.

20. A method according to claim 17 wherein the vaccine is formulated with one or more added adjuvants.

21. A method according to claim 20 wherein the adjuvant is selected to promote a Th1 T-cell immune response and/or to suppress a Th2 T-cell immune response in the mammal.

22. A method according to claim 17 wherein the cellular fraction is prepared from the whole said microorganism.

23. A method according to claim 17 wherein the cellular fraction is a sonicate.

24. A method according to claim 17 wherein the cellular fraction is filterable through a filter with a pore size of less than 0.60 μm.

25. A method according to claim 24 wherein the pore size is about 0.20 μm or less.

26. A method according to claim 17 wherein the microorganism is a microorganism that colonises the lung or respiratory tract.

27. A method according to claim 17 wherein the immune response is a systemic immune response.

28. A method according to claim 17 for the prophylaxis or treatment of an infection by the microorganism selected from the group consisting of lung, oral, nasal, oropharyngeal, nasalpharyngeal, pharyngeal, respiratory tract, digestive tract, vaginal, urinary tract, kidney, eye and skin infections.

29. A method according to claim 28 wherein the infection is a lung or respiratory tract infection.

30. A method according to claim 17 wherein the microorganism is a bacterial, fungal or yeast pathogen.

31. A method according to claim 30 wherein the microorganism is a bacterial pathogen selected from the group consisting of Non-typeable H. influenzae, Moraxella catarrhalis, S. pneumoniae, P. aeruginosa, H. influenzae type b, H. pylori, S. aureus, S. albus, C. pneumoniae, C. trachomatis, S. pyrogenes, E. coli species and Mycoplasma species.

32. A method according to claim 31 wherein the bacterial pathogen is selected from Non-typeable H. influenzae, S. pneumoniae and P. aeruginosa.

33. A method for prophylaxis or treatment of a disease or condition in a mammal associated with, or exacerbated by, infection by at least one pathogenic microorganism, the method comprising administering an effective amount of a cellular fraction of the microorganism to mucosa of the lungs of the mammal for generating an immune response against the microorganism, wherein the cellular fraction is essentially free of particulate matter and includes polyvalent soluble antigen from the microorganism.

34. A method according the claim 33 wherein the cellular fraction comprises cellular matter and the polyvalent soluble antigen.

35. A method according to claim 33 wherein the vaccine is formulated without any added adjuvants.

36. A method according to claim 33 wherein the vaccine is formulated with one or more added adjuvants.

37. A method according to claim 36 wherein the adjuvant is selected to promote a Th1 T-cell immune response and/or to suppress a Th2 T-cell immune response in the mammal.

38. A method according to claim 33 wherein the cellular fraction is prepared from the whole said microorganism.

39. A method according to claim 33 wherein the cellular fraction is a sonicate.

40. A method according to claim 33 wherein the cellular fraction is filterable through a filter with a pore size of less than 0.60 μm.

41. A method according to claim 40 wherein the pore size is less than 0.20 μm.

42. A method according to claim 33 wherein the microorganism is a microorganism that colonises the lung or respiratory tract.

43. A method according to claim 33 wherein the immune response is a systemic immune response.

44. A method according to claim 33 wherein the infection is an infection of the lung or respiratory tract.

45. A method according to claim 33 wherein the microorganism is a bacterial pathogen.

46. A method according to claim 45 wherein the bacterial pathogen is selected from the group consisting of H. influenzae type b, Non-typeable H. influenzae, S. pneumoniae and P. aeruginosa.

47. A method according to claim 33 wherein the disease or condition is selected from the group consisting of otitis media, pneumonia, chronic bronchitis, cystic fibrosis, asthma, lung conditions and superinfections following viral infection.
